(19) United States
(12) Patent Application Publication
(10) Pub. No.: US 2026/0083694 A1
(43) Pub. Date: Mar. 26, 2026

(54) COMPOSITION COMPRISING L-LEUCINE AND FISH OIL

(71) Applicant: ABIOGEN PHARMA S.P.A., Loc. Ospedaletto, Pisa (IT)

(72) Inventors: Mattia Sfriso, Istrana (TV) (IT); Mauro Bonini, Istrana (TV) (IT); Sara Feltrin, Istrana (TV) (IT); Lucia Florian, Istrana (TV) (IT)

(73) Assignee: ABIOGEN PHARMA S.P.A., Loc. Ospedaletto, Pisa (IT)

(21) Appl. No.: 19/109,400

(22) PCT Filed: Sep. 5, 2023
(86) PCT No.: PCT/IB2023/058778

§ 371 Date: Mar. 6, 2025

(30) Foreign Application Priority Data
Sep. 14, 2022 (IT) 102022000018804

(51) Int. Cl. A61K 31/198; A61K 9/00; A61K 35/00; A61K 35/60; A61K 35/747; A61K 47/26; A61K 47/36

(52) U.S. Cl. CPC A61K 31/198; A61K 9/0056; A61K 35/60; A61K 35/747; A61K 47/26; A61K 47/36; A61K 2035/115

(57) ABSTRACT

The invention concerns a composition comprising L-leucine and fish oil usable to supplement leucine and omega 3 in subjects in need thereof. The invention also concerns the use of the said composition in the treatment of muscle 5 tissue pathologies, in particular for the prevention of muscle pathologies characterised by loss of muscle mass and/or muscle strength, such as sarcopenia.

### FIELD OF THE INVENTION

The invention concerns a composition comprising L-leucine and fish oil that can be used to supplement leucine and omega 3 in subjects in need thereof.

The invention also concerns the use of the said composition in the treatment of muscle tissue pathologies, in particular for the prevention of muscle pathologies characterised by loss of muscle mass and/or muscle strength, such as sarcopenia.

### BACKGROUND ART

L-leucine is an amino acid belonging to the group of the so-called essential amino acids, i.e. those amino acids that cannot be synthesised by the organism and therefore must be taken with diet.

The main function of L-leucine is as a precursor of protein synthesis, being particularly important at the muscular level, because it directly stimulates myofibrillar muscle protein synthesis.

It has been discovered that, by increasing the synthesis of muscle proteins and therefore helping maintain muscle mass and strength, leucine can slow down muscle tissue deterioration.

Leucine is an aliphatic branched-chain amino acid, non-polar and poorly soluble in water. Two enantiomers of leucine can be identified: 2 (S)-amino-3-methylpentanoic acid and 2(R)-amino-3-methylpentanoic acid. From an optical point of view, these two molecules are defined as L-leucine and D-leucine. L-enantiomer is used by living beings to produce proteins.

Omega-3 also fulfils a beneficial function for muscle mass.

Omega 3 (n−3 PUFAs) are polyunsaturated fats, which are considered essential. Indeed, their precursor (alpha-linolenic acid, ALA) cannot be synthesised by the body and therefore it must be taken with food.

The main animal sources of omega 3 are fatty fish, such as anchovies, herring, mackerel, salmon, sardines, sturgeon, trout, cod, and tuna, and the oils that are prepared therefrom. These oils are especially rich in omega 3 EPA (eicosapentaenoic acid) and DHA (docosahexaenoic acid), which-once consumed in one's diet—are readily usable by the body.

Compositions including L-leucine and fish oil can be advantageously used for the supplementation of nutrients which are essential for muscles.

However, the fishy smell that is characteristic of fish oils, even in the presence of leucine, makes consumption thereof unappealing.

To overcome this problem encountered with fish oils, formulations have been proposed over time, for example coated by capsules, which are intended to facilitate the ingestion of omega 3, but unfortunately these solutions do not resolve the unpleasant fishy odour of the oils, which remains in the mouth even after ingestion, causing bad breath; in some cases the fishy odour can also be smelt on the skin.

To overcome the problem of the adverse organoleptic properties of fish oils, attempts have been made to use plant-based sources of Omega 3, such as walnuts, flaxseeds, chia seeds, and the oils thereof, in addition to soya oil. These sources are particularly rich in ALA, which—in order to be used by the human organism-must first be converted into EPA and DHA. However, some recent studies (Lane et al, 2014) have reported that, actually, very little ALA from plant sources is actually converted into EPA and DHA, or it is not converted at all.

Patent application EP3511010 describes an odourless fish oil, obtained by mixing gums, starch or methylcellulose with water at high temperatures (about 80-85° C.), to obtain a colloid which will then be mixed with the fish oil, in order to obtain an odourless emulsion. However, this preparation procedure is complex and requires energy expenditure to heat the mixture to high temperatures. Furthermore, in the industrial preparation of compositions comprising oils, problems are encountered relating to the processability of the mixtures. These processability problems are also encountered with leucine, as this amino acid is poorly soluble in water.

Compositions including leucine and fish oil therefore present processability problems, and even more when including gum, in the aim of improving the organoleptic properties of the product.

The object of the present invention is therefore to provide compositions comprising L-Leucine and fish oil which have pleasant organoleptic properties and which are adequately processable, so as to overcome the problems and limitations of the prior art.

### SUMMARY OF THE INVENTION

This object has been achieved by a composition comprising L-leucine, fish oil, polyoxyethylene sorbitan monooleate (or polysorbate), and at least one gum, as defined in the appended claims.

The present invention also concerns a food or a dietary supplement comprising the composition according to the invention and suitable food carriers, preferably in the form of powder or granules to be reconstituted in water for oral administration.

In another aspect, the invention concerns the use of the composition, as well as of the food or supplement according to the invention for the treatment of muscle tissue pathologies, such as muscle atrophy or sarcopenia.

### DETAILED DESCRIPTION OF THE INVENTION

Therefore, the invention concerns a composition comprising L-leucine, fish oil, polyoxyethylene sorbitan monooleate, and at least one gum, wherein the weight ratio of gum to fish oil is higher than 0.3 and lower than 0.8, and wherein the weight ratio of polyoxyethylene sorbitan monooleate to L-leucine is higher than 0.01 and lower than 0.06.

For the purposes of the present invention, the term “L-leucine” refers indifferently to the enantiomer L-leucine either pure or mixed with D-leucine, preferably in a lower concentration than L-leucine, and more preferably in trace amounts at the most.

Preferably, the L-leucine according to the present invention is pure L-leucine, i.e. not comprising the enantiomer D-leucine.

Fish oils which are suitable for use in the composition according to the present invention include, among others, fish oils obtained from anchovies, herring, mackerel, salmon, sardines, sturgeon, trout, cod and tuna, obtained according to prior art methods.

The term “gum” generally denotes a natural or synthetic polymeric material, characterised by high elasticity. For the purposes of the present invention, the term “gum” means a natural gum, comprising or consisting of polysaccharides.

Examples of gums that can be used in the present invention are gum arabic, acacia gum, xanthan gum, ghatti gum, tragacanth gum, fenugreek gum, mesquite gum, or a mixture thereof.

Preferably, said at least one gum is chosen from gum arabic, xanthan gum, mesquite gum, and mixtures thereof.

In preferred embodiments, the composition of the invention comprises gum arabic or a mixture of gum arabic and xanthan gum.

Preferably, the weight ratio between gum and fish oil is between 0.4 and 0.7, more preferably 0.5 and 0.6.

Preferably, the weight ratio between polyoxyethylene sorbitan monooleate and L-leucine is between 0.02 and 0.05, more preferably between 0.02 and 0.03.

Indeed, it has surprisingly been found that the selection of these specific ingredients, in the quantities and ratios specified, allows to achieve satisfactory processability in the production stage, and likewise pleasant organoleptic properties, adequately masking the odour and flavour of the fish oil.

Surprisingly, it has also been observed that use of a water-soluble aroma alone is not able to reduce the fishy odour and taste of the oil, while the addition of fat-soluble aromas (lipoaromas) to the composition are capable of improving the organoleptic properties thereof, further increasing palatability. Preferably, therefore, the composition according to the invention further comprises at least one fat-soluble aroma, optionally in combination with at least one water-soluble aroma.

Aromas employable in preferred compositions according to the present invention include orange+carrot+lemon (ACE) lipoaroma, mint lipoaroma, mint aroma, lemon aroma, and mixtures thereof.

Preferably, the composition further comprises at least one sweetener, to further improve the palatability thereof.

The composition can optionally also comprise excipients.

The term “excipient” means a compound or a mixture of compounds which are suitable for pharmaceutical or food use, respectively. For example, an excipient for use in a pharmaceutical or food formulation should not generally cause an adverse response in a subject, nor should it significantly inhibit the efficacy of the cholecalciferol contained therein.

Suitable excipients include acidifiers, acidity regulators, anti-caking agents, antioxidants, bulking agents, resistance agents, gelling agents, coating agents, modified starches, sequestering agents, thickeners, sweeteners, diluents, disaggregating agents, glidants, colorants, binders, lubricants, stabilisers, adsorbents, humectants, flavourings, filmogenic agents, emulsifiers, wetting agents, release retardants, and mixtures thereof.

In some embodiments, the composition according to the invention consists essentially of L-leucine, fish oil, polysorbate, and at least one gum. The expression “consists essentially of” means that additional components in the composition do not interfere with the action and properties of the ingredients specified.

In other embodiments, the composition according to the invention consists of L-leucine, fish oil, polysorbate, and at least one gum, and suitable excipients. The expression “consists of” means that there are no additional ingredients present in the composition.

It should also be understood that all aspects stated above as favourable and advantageous for the composition and the components thereof should be deemed likewise preferable and advantageous also for these embodiments defined, respectively, with the expressions “consists essentially of” and “consists of”.

The composition of the present invention can be prepared using prior art methods. Indeed, for administration via oral route, the components can, for example, be mixed as they are, or with one or more excipients, either sealed in soft-gel capsules or in solid form, such as a tablet, mini-tablet, micro-tablet, granule, micro-granule, pellet, multiparticulate, micronised particulate, powder, or in the form of a solution, emulsion, gel, ampoule, or of drops or sprays.

Preferably, the composition of the invention is in solid form.

When the composition is in solid form, it may also contain one or more coating agents. The term “coating agent” refers to a film-forming polymer capable of coating the surface of the solid form, thereby forming an external layer that not only allows the release of the ingredients to be modulated, but also further masks the flavour of the fish oil and the leucine.

Suitable film-forming polymers are cellulose acetate phthalate, cellulose acetate succinate, hydroxypropyl methylcellulose phthalate, hydroxypropyl methylcellulose acetate succinate, polyvinyl acetate phthalate, hydroxyethyl cellulose phthalate, cellulose acetate tetrahydro phthalate, copolymers of methacrylate-methacrylic acid, copolymers of methyl methacrylate-methacrylic acid, sodium alginate, stearic acid, cellulose acetate trimellitate, shellac, hydroxypropyl methylcellulose, hydroxypropyl cellulose, hydroxyethyl cellulose, ethyl cellulose, cross-linked carboxymethyl cellulose and the salts thereof, polyvinyl alcohol, pH-independent polymethacrylates, polyvinyl acetate, polyvinyl pyrrolidone, fatty acids, medium-chain triglycerides, or a mixture thereof.

Preferably, the coating agent comprises ethyl cellulose, hydroxypropyl cellulose, hydroxypropyl methylcellulose, cellulose, fatty acids, medium-chain triglycerides, or a mixture thereof.

When the composition in solid form comprises at least one coating agent, this composition can advantageously be prepared by means of a process comprising the following steps:
i) weighing the L-leucine, sieving and then leaving the latter for approximately 15 minutes in a mixer;
ii) spraying onto the L-leucine an emulsion comprising gum, fish oil, polyoxyethylene sorbitan monooleate, water, and optionally lipoaroma, and mixing,
iii) adding an aqueous solution of a coating agent under stirring and allowing the thus coated solid form to dry.

In all the embodiments, the composition according to the invention was found to be palatable and pleasant to the taste, with excellent organoleptic characteristics, stable and easy to prepare and produce.

The present invention also concerns a dietary supplement or a food comprising the composition according to the invention and suitable food carriers.

Preferably, suitable food carriers are sweeteners, stabilisers, thickeners, acidifiers, colourings, aromas/, bulking agents, and mixtures thereof.

Preferably, the supplement or food according to the invention comprises bulking agents such as calcium phosphates, calcium carbonates, mannitol, maltodextrins, or a mixture thereof.

Preferably, the dietary supplement or food according to the invention is in solid form, such as tablet, mini-tablet, micro-tablet, granule, micro-granule, pellet, multi-particulate, micronised particulate, powder, or a mixture thereof.

Advantageously, the supplement or food according to the invention can also contain probiotics, vitamins, mineral salts, carbohydrates, proteins, phytonutrients, and in general any other ingredient with a supplementation function.

In a particularly advantageous embodiment, the supplement or food according to the invention comprises at least one probiotic.

Preferably, the probiotics suitable for the supplement or food according to the invention belong to the Lactobacillus or Bifidobacterium genus.

Even more preferably, said probiotics belong to the Lactobacillus genus.

In particularly preferred embodiments, the supplement or food according to the invention comprises at least one probiotic of the L. paracasei species.

In a particularly preferred embodiment, the supplement or food comprises L-leucine, fish oil, polyoxyethylene sorbitan monooleate, gum, and a probiotic belonging to the Lactobacillus genus, wherein:
gum and fish oil have a weight ratio higher than 0.3 and lower than 0.8, preferably from 0.4 to 0.7, more preferably from 0.4 to 0.5, and
polyoxyethylene sorbitan monooleate and L-leucine are in weight ratio higher than 0.01 and lower than 0.06, preferably from 0.02 to 0.05, more preferably from 0.02 to 0.03.

In a particularly advantageous embodiment of the invention, said supplement or food is in powder form and comprises lipoaroma, L-leucine, gum arabic, fish oil, polyoxyethylene sorbitan monooleate, a coating agent, preferably comprising hydroxypropyl cellulose, hydroxypropyl methylcellulose or a mixture thereof, a probiotic of the Lactobacillus genus, sucralose, water-soluble aroma, citric acid, calcium phosphates, xanthan gum, and mannitol.

In preferred embodiments, the composition according to the invention and likewise the dietary supplement or food according to the invention, is in unit dose form.

Preferably, the said unit dose comprises 2-10 g of composition, more preferably 4-6 g, most preferably about 5 g.

For example, the composition according to the invention, and likewise the dietary supplement or food according to the invention, can be in unit dose form in powder or granules.

Advantageously, said unit dose in powder or granules is contained in a single-dose sachet.

In another aspect, the present invention concerns the use of the composition, and likewise the supplement or food, for the treatment of muscle tissue pathologies, such as muscle atrophy or sarcopenia. For the purposes of the present invention, the term “treatment” means the administration of the composition, food or supplement according to the present invention to a subject at risk of, or already affected from, muscle tissue pathology, with the aim of improving the overall condition of the muscle tissue thereof, as well as for the purpose of slowing down, alleviating, reducing, and/or preventing any alteration in the functioning of their muscle tissue, in particular to prevent and counteract the loss of muscle mass and muscle strength.

Preferably, said muscle tissue pathology is muscle atrophy or sarcopenia. The risk factors for development or worsening of a muscle tissue pathology include physiological ageing.

Sarcopenia is a syndrome characterised by the progressive and generalised loss of skeletal muscle's mass and strength (muscle failure) and it is closely related to physical disabilities, poor quality of life, and death.

In clinical practice, case identification can begin when the patient reports symptoms or signs of sarcopenia (i.e., falls, feeling weak, slow walking speed, difficulty getting up from a chair, or weight loss/muscle atrophy).

Sarcopenia can be diagnosed by standardised questionnaires, or by measurement of grip strength using a calibrated hand dynamometer, under well defined test conditions, with interpretive data from appropriate reference populations.

Preferably, in the treatment of muscle tissue pathologies, the composition, and likewise the supplement or food, must be administered by oral, buccal or sublingual route, preferably by oral route.

More preferably, the composition, and likewise the supplement or food, is in solid form and must be reconstituted prior to administration and more preferably must be dissolved in water prior to administration.

In preferred embodiments, said solid form is powder or granules.

More preferably, in the treatment of muscle tissue pathologies, the composition, and likewise the supplement or food, must be administered at a sufficient daily dose to provide 1-5 g L-leucine and up to 1 g fish oil.

It should be understood that all the possible combinations of the preferred aspects of the composition and the supplement or food containing the said composition, and likewise the respective uses, as stated above, are described herein and therefore are also preferred.

It should also be understood that all the aspects identified as preferred and advantageous for the composition according to the invention must be considered similarly preferred and advantageous also for the preparation and uses of the said composition and likewise the dietary supplement and the food.

The following illustrative, non-limiting examples are provided to support the invention.

### Examples

### Example 1. Processability Assessment

Various compositions comprising gum arabic or mesquite gum and fish oil were prepared, and it was determined which ones provided acceptable processability. The results of the tests are summarised in table 1a below:

TABLE 1a

| | Test 1 | Test 2 | Test 3 | Test 4 | Test 5 |
|---|---|---|---|---|---|
| Gum/fish oil | 0.53 | 0.70 | 0.42 | 0.80 | 0.30 |
| weight ratio | | | | | |
| Processability | Acceptable | Acceptable | Acceptable | Not acceptable | Not acceptable |

As it is evident, the tests have shown that the weight ratio between gum and fish oil must be higher than 0.3 and less than 0.8 to be acceptable. Indeed, when the ratio is higher than the value of 0.7, the viscosity of the solution increases and at 0.8 the solution becomes almost a gel and can no longer be sprayed. Conversely, when the value falls below 0.3, the solution is no longer stable.

Tests were then conducted at different weight ratios of polyoxyethylene sorbitan monooleate and leucine. The tests conducted are shown below in table 1b:

TABLE 1b

| | Test 1 | Test 2 | Test 3 | Test 4 | Test 5 |
|---|---|---|---|---|---|
| Polyoxyethylene | 0.023 | 0.030 | 0.010 | 0.05 | 0.060 |
| sorbitan monooleate/ | | | | | |
| L-leucine weight ratio | | | | | |
| Processability | Acceptable | Acceptable | Not acceptable | Acceptable | Not acceptable |

As can be seen from the tests carried out, the ratio between polyoxyethylene sorbitan monooleate and leucine must be higher than 0.01 and lower than 0.06 to have acceptable processability. If the value falls below 0.01, a “cap effect” is generated, with all the leucine on the surface rather than being dispersed in the aqueous solution. Conversely, a ratio higher than 0.06 would generate a foamy solution.

### Example 2. Assessment of Organoleptic Properties

Once the acceptable ranges of gum/fish oil and polyoxyethylene sorbitan monooleate/L-leucine ratios had been determined in order to achieve good processability during the industrial production method, trials were performed with different types and combinations of aromas to further mask the odour and the taste of the fish oil.

Typical formulations were tested that had a gum/fish oil ratio of approximately 0.53 and a polyoxyethylene sorbitan monooleate/L-leucine ratio of approximately 0.023, with different types of aromas combinations, as shown below in table 2:

TABLE 2

| Ingredient | Test 1 | Test 2 | Test 3 | Test 4 |
|---|---|---|---|---|
| Water-soluble | Absent | Present | Absent | Present |
| aroma | | | | |
| Fat-soluble aroma | Absent | Absent | Present | Present |
| (lipoaroma) | | | | |
| Organoleptic | Not | Not | Acceptable | Acceptable |
| properties | acceptable | acceptable | (odour) | (odour and |
| | | | | taste) |

The tests showed that the lipoaroma alone was able to mask both the odour and the taste of the fish oil. Furthermore, even more pleasant results were obtained from a palatability point of view by adding a water-soluble aroma in addition to the fat-soluble aroma.

### Example 3. Preparation of a Composition According to the Invention

A composition was prepared as shown below in table 3:

TABLE 3

| Components | Weight (grams) |
|---|---|
| Lipoaroma | 0.12 |
| L-leucine | 2.50 |
| Gum arabic | 0.31 |
| Fish oil | 0.59 |
| Polyoxyethylene sorbitan monooleate | 0.06 |
| Sucralose | 0.02 |
| Water-soluble aroma | 0.20 |
| Citric acid | 0.10 |
| Calcium phosphates | 0.40 |
| Xanthan gum | 0.10 |
| Mannitol | 0.44 |

Said composition was prepared by weighing the appropriate amount of L-leucine, and then sieving, mixing for 15 minutes in a mixer, and storing the L-leucine in a suitable container. A homogeneous emulsion comprising gum arabic, fish oil, polyoxyethylene sorbitan monooleate, fat-soluble aroma, and water was then sprayed onto the leucine.

This step was carried out with constant, homogeneous movement of the obtained mixture.

Finally, once appropriately weighed, the remaining ingredients were added and mixed until the composition was obtained in powder or granule form.

### Example 4. Preparation of a Composition According to the Invention

A composition was prepared as shown below in table 4:

TABLE 4

| Components | Weight (grams) |
|---|---|
| Lipoaroma | 0.12 |
| L-leucine | 2.50 |
| Gum arabic | 0.31 |
| Fish oil | 0.59 |
| Polyoxyethylene sorbitan monooleate | 0.06 |
| Coating agent (comprising hydroxypropyl cellulose, | 0.23 |
| and hydroxypropyl methylcellulose) | |
| Sucralose | 0.02 |
| Water-soluble aroma | 0.20 |
| Citric acid | 0.10 |
| Calcium phosphates | 0.40 |
| Xanthan gum | 0.10 |
| Mannitol | 0.44 |

Said composition was prepared by weighing the appropriate amount of L-leucine, and then sieving, mixing for 15 minutes in a mixer, and storing the L-leucine in a suitable container. A homogeneous emulsion comprising gum arabic, fish oil, polyoxyethylene sorbitan monooleate, fat-soluble aroma/flavouring, and water was then sprayed onto the leucine. This step was carried out with constant, homogeneous movement of the obtained mixture.

Subsequently, an aqueous solution of coating agent (including hydroxypropyl cellulose and hydroxypropyl methylcellulose) was added to said mixture, also under constant homogeneous movement.

Finally, once appropriately weighed, the remaining ingredients were added and mixed until the composition was obtained in powder or granule form.

### Example 5. Preparation of a Food According to the Invention

A food was prepared including the composition of Example 4 and a probiotic, as shown below in table 5:

TABLE 5

| Components | Weight (grams) |
|---|---|
| Lipoaroma | 0.12 |
| L-leucine | 2.50 |
| Gum arabic | 0.31 |
| Fish oil | 0.59 |
| Polyoxyethylene sorbitan monooleate | 0.06 |
| Coating agent (comprising hydroxypropyl cellulose, | 0.23 |
| and hydroxypropyl methylcellulose) | |
| Probiotic (Lactobacillus 300 billion/g) | 0.13 |
| Sucralose | 0.02 |
| Water-soluble aroma | 0.20 |
| Citric acid | 0.10 |
| Calcium phosphates | 0.40 |
| Xanthan gum | 0.10 |
| Mannitol | 0.44 |
| TOTAL | 5.20 |

Said food was prepared by weighing the appropriate amount of L-leucine, and then sieving, mixing for 15 minutes in a mixer, and storing the L-leucine in a suitable container. A homogeneous emulsion composed of gum arabic, fish oil, polyoxyethylene sorbitan monooleate, lipoaroma and water was then sprayed onto the leucine. This step was carried out with constant, homogeneous movement of the obtained mixture.

Subsequently, an aqueous solution of coating agent (including hydroxypropyl cellulose and hydroxypropyl methylcellulose) was added to the said mixture, still under constant homogeneous movement.

Finally, once appropriately weighed and mixed, the remaining ingredients were added, taking care to add the probiotic at the end to minimise mechanical stress.

Either the composition according to the invention in Examples 3 and 4 and the food in Example 5 were found to be palatable and pleasant to the taste, with excellent organoleptic characteristics, and were stable and easy to prepare and produce.

1. Composition comprising L-leucine, fish oil, polyoxyethylene sorbitan monooleate, and at least one gum, wherein the weight ratio of gum to fish oil is higher than 0.3 and lower than 0.8, and wherein the weight ratio of polyoxyethylene sorbitan monooleate to L-leucine is higher than 0.01 and lower than 0.06.

2. The composition of claim 1, wherein said at least one gum is selected from Gum arabic, xanthan gum, mesquite gum, and their mixtures.

3. The composition of claim 1, wherein the weight ratio of gum to fish oil is 0.4 to 0.7.

4. The composition of claim 1, wherein the weight ratio of polyoxyethylene sorbitan monooleate to L-leucine is 0.02 to 0.05, preferably 0.02 to 0.03.

5. The composition of claim 1, further comprising a fat-soluble aroma and, optionally, a water-soluble aroma.

6. The composition of claim 1, further comprising a coating agent.

7. Dietary supplement or food comprising the composition of claim 1 and suitable food carriers.

8. The dietary supplement or food of claim 7, further comprising at least one probiotic.

9. The composition of claim 1 or a dietary supplement or food comprising the composition of claim 1 and suitable food carrier, in solid form selected from tablet, mini-tablet, micro-tablet, granule, micro-granule, pellet, multi-particulate, micronized particulate, and powder.

10. The composition, dietary supplement or food of claim 9, in the form of a unit dose comprising 2-10 g of a composition comprising
L-leucine,
fish oil,
polyoxyethylene sorbitan monooleate, and at least one gum,
wherein the weight ratio of gum to fish oil is higher than 0.3 and lower than 0.8, and wherein the weight ratio of polyoxyethylene sorbitan monooleate to L-leucine is higher than 0.01 and lower than 0.06.

11. A method of treating muscle pathologies in a patient in need thereof, the method comprising
administering to said patient a therapeutically effective amount of the composition of claim 1 or a dietary supplement or food comprising the composition of claim 1 and suitable food carriers.

12. The method of claim 11, wherein the muscle tissue pathology is sarcopenia.

13. The method of claim 11, wherein a composition comprising
L-leucine,
fish oil,
polyoxyethylene sorbitan monooleate, and at least one gum,
optionally with suitable food carriers;
wherein the weight ratio of gum to fish oil is higher than 0.3 and lower than 0.8, and wherein the weight ratio of polyoxyethylene sorbitan monooleate to L-leucine is higher than 0.01 and lower than 0.06 is to be administered via oral, buccal or sublingual route.

14. The method of claim 11, wherein a composition comprising
L-leucine,
fish oil,
polyoxyethylene sorbitan monooleate, and at least one gum,
optionally with suitable food carriers;
wherein the weight ratio of gum to fish oil is higher than 0.3 and lower than 0.8, and wherein the weight ratio of polyoxyethylene sorbitan monooleate to L-leucine is higher than 0.01 and lower than 0.06, is to be administered at a daily dose so as to provide 1-5 g of L-leucine and up to 1 g of fish oil.

15. The composition of claim 2, wherein said at least one gum is Arabic gum or a mixture of Arabic gum and xanthan gum.

16. The composition of claim 3, wherein the weight ratio of gum to fish oil is 0.5 to 0.6.

17. The composition of claim 4, wherein the weight ratio of polyoxyethylene sorbitan monooleate to L-leucine is 0.02 to 0.03.

18. The composition of claim 6, wherein the coating agent comprises ethylcellulose, hydroxypropylcellulose, hydroxypropylmethyl cellulose, cellulose, fatty acids, medium chain triglycerides or a mixture thereof.

19. The composition or the dietary food of claim 9, in the form of a unit dose comprising 4-6 g of a composition comprising
L-leucine,
fish oil,
polyoxyethylene sorbitan monooleate, and at least one gum,
wherein the weight ratio of gum to fish oil is higher than 0.3 and lower than 0.8, and wherein the weight ratio of polyoxyethylene sorbitan monooleate to L-leucine is higher than 0.01 and lower than 0.06.
